(19) United States
(12) Patent Application Publication
(10) Pub. No.: US 2007/0218505 A1
(43) Pub. Date: Sep. 20, 2007

(54) IDENTIFICATION OF BIOMOLECULES THROUGH EXPRESSION PATTERNS IN MASS SPECTROMETRY

(76) Inventor: Paul Kearney, Montreal (CA)

Correspondence Address: TORYS LLP, 79 WELLINGTON ST. WEST, SUITE 3000, TORONTO, ON, M5K 1N2, CA

(21) Appl. No.: 11/686,247

(22) Filed: Mar. 14, 2007

Related U.S. Application Data
(60) Provisional application No. 60/781,720, filed on Mar. 14, 2006.

(51) Int. Cl. G06F 19/00; G01N 33/53

(52) U.S. Cl. 435/7.1; 702/19

(57) ABSTRACT

Systems, methods, apparatus, and computer programming useful in identifying proteins, peptides, carbohydrates, and other biomolecules, or for a validation of an identification of proteins, peptides, carbohydrates, and other biomolecules, is described. In particular, the invention provides systems, methods, apparatus, and programming useful for identifying proteins and other precursor biomolecules using expression patterns associated with peptides or other biomolecule fragments expressed from analyte samples, and data representing such expression patterns, and for determining and improving confidence levels associated with identification of precursor biomolecules using such methods, through the correlation of expression patterns for fragments associated with precursor biomolecules.

### CROSS-REFERENCE TO RELATED APPLICATIONS AND INCORPORATION BY REFERENCE

This application claims the benefit of U.S. provisional patent application Ser. No. 60/781,720, filed 14 Mar. 2006 and entitled “AUTOMATED IDENTIFICATION OF BIOMOLECULES THROUGH EXPRESSION PATTERNS IN MASS SPECTROMETRY”, the entire contents of which, including any appendices, is incorporated by reference.

This application is related to (i) U.S. provisional patent application Ser. No. 60/691,414, filed Jun. 16, 2005 and entitled “VIRTUAL MASS SPECTROMETRY”, the entire contents of which, including any appendices, is incorporated herein by reference, and (ii) U.S. non-provisional patent application Ser. No. 10/293,076, filed 13 Nov. 2002 and entitled “Mass Intensity Profiling System and Uses Thereof”, the entire contents of which, including any appendices, is incorporated herein by reference.

The following are also incorporated by reference:
Cohen, J., Cohen P., West, S. G., and Aiken, L. S. (2003), Applied multiple regression/correlation analysis for the behavioral sciences (3rd ed.), Hillsdale, N.J.: Lawrence Erlbaum Associates
Jimmy K. Eng, Ashley L. McCormack and John R. Yates, III An approach to correlate tandem mass spectral data of peptides with amino acid sequences in a protein database, JASMS, Volume 5, Issue 11, November 1994, Pages 976-989;
Pappin D. J., Hojrup, P., Bleasby, A. J., Rapid identification of proteins by peptide-mass fingerprinting, Curr Biol. 3 (6), 327-32, 1993; and Adkins, J. N., Monroe, M. E., Auberry, K. J., Yufeng, S., et al., A proteomic study of the HUPO Plasma Proteome Project's pilot samples using an accurate mass and time tag strategy, Proteomics, 5, 3454-3466, 2005;
Peng, Junmin. et al. Evaluation of multidimensional chromatography coupled with tandem mass spectrometry (LC/LC-MS/MS) for large-scale protein analysis: The yeast proteome, Journal of Proteome Research, 2, 43-50, 2003;
Gygi, S P, Rist, B, Gerber, S A, Turecek, F, Gelb, M H, and Aebersold, R. 1999. Quantitative analysis of complex protein mixtures using isotope-coded affinity tags. Nature Biotechnology, 17:994-999;
J. Lamerz et al., Correlation-associated peptide networks of human cerebrospinal fluid, Proteomics, 5, 2789-2798, 2005;
Laemmli, Nature 1970, 227:680-685;
Washburn et al., Nat. Biotechnol. 2001, 19:242-7; Schagger et al., Anal. Biochem. 1991, 199:223-31;
Godovac-Zimmermann et al. (2001) Mass Spectrom. Rev. 20: 1-57 (PMID: 10344271);
Gygi et al., (2000) Proc. Natl. Acad. Sci. U.S.A. 97: 9390-9395 (PMID: 10920198) [hereinafter “Gygi et al. II”];
Reinders et al., 2004 Proteomics 4: 3686-703;
Aebersold et al., 2003 Nature 422: 198-207;
Garey, Michael R. and Johnson, David S., (1979) Computers and Intractability: A Guide to the Theory of NP-Completeness, W. H. Freeman; and
Brucella abortus, Proteome Research, 2007; ASAP Article; DOI: 10.1021/pr060636a.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent files or records, but otherwise reserves all copyrights whatsoever.

### FIELD OF INVENTION

The invention relates to the fields of mass spectrometry and the identification of polypeptides and other biomolecules.

### SUMMARY OF THE INVENTION

Mass spectrometry and related techniques have become important tools in the analysis of proteins, peptides, carbohydrates, and other biomolecules and biomolecule fragments, the understanding and identification of which are important in a wide variety of fields. For example, proteomic research programs typically include the identification of protein content of any given tissue, cell, subcellular organelle or bodily fluid, their isoforms, splice variants post-translation modifications, interacting partners, and higher-order complexes under different conditions. In other applications, samples from different study conditions are compared such as healthy, diseased and disease-treated with the intent of identifying proteins that are differentially expressed between the conditions. These proteins can be developed into therapeutics, biomarkers or diagnostics of human disease. Such analyses also aid in the fundamental understanding of disease and disease treatment. Indeed, many activities, innovations and decisions in basic biological research and pharmaceutical development depend on the accuracy of protein identification.

In one aspect, for example, the invention provides computer-usable media comprising computer-readable programming code adapted for causing a computer or other data processor to access data representing a plurality of expression patterns of peptides or other biomolecule fragments expressed from one or more samples and, using the accessed data, to identify or otherwise associate at least one protein or other biomolecule associated with the plurality of fragment expression patterns, and to determine coefficients useable for measuring correlations between the pluralities of expression patterns identified as associated with the various biomolecules. Such coefficients can be used, for example, in conjunction with, or without, other data to identify relatively high-confidence and a relatively low-confidence associations of fragments with precursor biomolecules.

Thus for example coefficients indicating a relatively low confidence in an association of a peptide or other biomolecule fragment with a protein or other biomolecule can be used to ensure that the association is not considered in subsequent analyses, or is at least identified as indicating a less-reliable identification and used accordingly in subsequent analyses. Furthermore such coefficients representing the correlation of peptide or biomolecule fragments matched to homologous or closely related biomolecules can be used to more accurately interpret the identification data and resolve between previously indistinguishable biomolecules or proteins.

The use of stored data sets representing previously-conducted analyses may be useful, for example, in confirming or improving the results of prior analyses. Stored data sets may be accessed from memory associated with the processor, as for example as a part of a computer adapted for controlling a mass spectrometer instrument, from a data base accessed locally or for from a local network source, as for example over a local area network (LAN), or remotely over a public or private electronics communications network (ECN) such as the internet or a private subscription service.

Thus, in an aspect of the invention there is a method useful in an identification of proteins. The method may be performed by a data processor and comprise: accessing data representing a plurality of expression patterns of peptides expressed from one or more samples; using the accessed data, identifying at least one protein associated with the plurality of peptide expression patterns; selecting a correlation coefficient useable for determining a correlation between each at least one protein and a plurality of expression patterns of peptides identified as associated therewith; and using at least the correlation coefficient, identifying at least one of a relatively high-confidence association and at least one of a relatively low-confidence association of precursor proteins with the peptides expressed from the one or more samples.

The correlation coefficient may include a correlation threshold value and a coverage threshold value. The identifying the at least one relatively high-confidence and low confidence associations of precursor proteins may include: identifying a largest subset of the plurality of expression patterns associated with the each at least one protein, the subset having pairwise correlation above the correlation threshold value; and identifying the each at least one protein as (i) a at least one relatively high-confidence association of precursor proteins if the subset size is greater or equal to the coverage threshold value, and (ii) a at least one relatively low-confidence association of precursor proteins if the subset size is small than the coverage threshold value.

The method may further comprise accessing second data representing randomized expression patterns of peptides. It may further comprise using at least the correlation coefficient, identifying from the second data at least one of a relatively high-confidence by-chance association and at least one of a relatively low-confidence by-chance association of the at least one proteins with the peptide expressed from the one or more samples. This identifying from the second data may be by: identifying in the second data a largest subset of the plurality of expression patterns by-chance associated with the each at least one protein, the subset having pairwise correlation above the correlation threshold value; and identifying the each at least one protein as (i) a at least one relatively high-confidence by-chance association if the subset size is greater or equal to the coverage threshold value, and (ii) a at least one relatively low-confidence by-chance association if the subset size is small than the coverage threshold value.

The method may further comprise determining a false positive rate as a ratio of a total of the at least one relatively high-confidence association of the precursor proteins over a total of the at least one relatively high-confidence by-chance association of the at least one proteins with the peptide expressed from the one or more samples. The method may further comprise evaluating whether the false positive rate is unacceptable, and if it is unacceptable, then selecting a new correlation threshold to replace the correlation threshold for use in repeating the said identifying steps until the false positive rate is acceptable.

The expression patterns may be obtained by liquid-chromatography/mass spectroscopy (LC-MS) analysis. The data relating to each expression pattern may be obtained by digesting a corresponding peptide with a protease. The accessing data representing the pluralities of expression patterns of peptides may comprise accessing data obtained using mass spectrometry. The accessing data representing the pluralities of expression patterns samples may comprise accessing data obtained using virtual mass spectrometry. The data representing the plurality of expression patterns of peptides expressed from the one or more samples may be accessed at least in part from real time analysis by a mass spectroscopy device associated with the processor.

The data representing a plurality of expression patterns of peptides expressed from one or more samples may be accessed at least in part from a stored data set. The stored data set may be stored in persistent media associated with the data processor. The stored data set may be accessed via a public communications network. The correlation may be between expression patterns obtained from a plurality of samples, with at least two of the samples collected from different subjects. The correlation may be between expression patterns from a plurality of samples, with at least two of the samples collected from a same subject at different times.

In another aspect of the invention, there is a method of validating a biomolecule identification from a plurality of peptides. The method may comprise: using at least an assignment of the plurality of peptides to at least one precursor biomolecule from a set of peptide expression profiles, determining a correlation coefficient for correlating the assignment of the plurality of peptides to the at least one precursor biomolecule within a false positive identification rate; and validating the biomolecule identification based on the assignment, if the biomolecule identification is correlated to one or more of the at least one precursor biomolecule within the false positive identification rate.

The false positive identification rate may be determined as a function of an expected random correlation between the plurality of peptides to the at least one biomolecule within the set of peptide expression profiles.

The expected random correlation may be a total number of expected false identifications based on the at least one biomolecule. The false positive identification rate may be determined as a ratio of the total number of expected false identifications over a total number of identifiable biomolecules. The total number of identifiable biomolecules may be based on the at least one biomolecule.

The correlation coefficient may comprise a correlation threshold and a coverage threshold. The total number of identifiable biomolecules may be determined by, for each of the at least one biomolecule, incrementing the total number of identifiable biomolecules if, in the set of peptide expression profiles, a largest subset of peptide assignment to the each at least one biomolecule has pairwise correlation above the correlation threshold and the subset has a size above the coverage threshold. The total number of expect false identifications may be determined by, for each of the at least one biomolecule, incrementing the total number of expected false identifications if, in a randomized set of peptide expression profiles, another largest subset of peptide assignment to the each at least one biomolecule has pairwise correlation above the correlation threshold and the subset has a size above the coverage threshold. The randomized set of peptide expression profiles may be generated from the set of peptide expression profiles.

The correlation coefficient may be selected on the basis of the false positive identification rate. The biomolecule may be a protein. The correlation coefficient may be selected from a plurality of test correlation coefficients, each of the test correlation coefficients being used to calculate a respective test false identification rate in the same manner that the correlation coefficient is used to determine the false positive identification rate. The test correlation coefficient having a test false identification rate that is closest within the false positive identification rate may be selected as the correlation coefficient.

The correlation coefficient may be selected by initially selecting a test correlation coefficient to determine a test false identification rate in the same manner that the correlation coefficient is used to determine the false positive identification rate. If the test false identification rate is not within the false positive identification rate, the method may iteratively adjust the test correlation coefficient until the test false identification rate is within the false positive identification rate, and then selecting the test correlation coefficient as the false positive identification rate.

In a further aspect of the invention, there is a computer usable medium having computer readable code embodied therein. The computer readable code may cause a computer to: access data representing a plurality of expression patterns of peptides expressed from one or more samples; using the accessed data, identify at least one protein associated with the plurality of peptide expression patterns. The computer readable code may further causes the computer to select a correlation coefficient useable for determining a correlation between each at least one protein and a plurality of expression patterns of peptides identified as associated therewith, the correlation coefficient having a correlation threshold value and a coverage threshold value. The computer readable code may further causes the computer to, using at least the correlation coefficient, identify at least one of a relatively high-confidence association and at least one of a relatively low-confidence association of precursor proteins with the peptides expressed from the one or more samples, by: identifying a largest subset of the plurality of expression patterns associated with the each at least one protein, the subset having pairwise correlation above the correlation threshold value; and identifying the each at least one protein as (i) a at least one relatively high-confidence association of precursor proteins if the subset size is greater or equal to the coverage threshold value, and (ii) a at least one relatively low-confidence association of precursor proteins if the subset size is small than the coverage threshold value.

The computer readable code may further causes the computer to access second data representing randomized expression patterns of peptides. The computer readable code may further causes the computer to, using at least the correlation coefficient, identify from the second data at least one of a relatively high-confidence by-chance association and at least one of a relatively low-confidence by-chance association of the at least one proteins with the peptide expressed from the one or more samples. The identify from the second data may be by: identifying in the second data a largest subset of the plurality of expression patterns by-chance associated with the each at least one protein, the subset having pairwise correlation above the correlation threshold value, and identifying the each at least one protein as (i) a at least one relatively high-confidence by-chance association if the subset size is greater or equal to the coverage threshold value, and (ii) a at least one relatively low-confidence by-chance association if the subset size is small than the coverage threshold value.

The computer readable code may further causes the computer to determine a false positive rate as a ratio of a total of the at least one relatively high-confidence association of the precursor proteins over a total of the at least one relatively high-confidence by-chance association of the at least one proteins with the peptide expressed from the one or more samples. The computer readable code may further causes the computer to evaluate whether the false positive rate is unacceptable, and if it is unacceptable, then selecting a new correlation threshold to replace the correlation threshold for use in repeating the said identifying steps until the false positive rate is acceptable.

In another aspect, there is a method for improving and measuring the accuracy of protein identification using peptide expression profiles. The method may comprise: providing a plurality of peptide-to-protein assignments; providing an expression profile over a plurality of samples for a plurality of peptides; for a plurality of correlation coefficient threshold and peptide coverage threshold pairs, determine the false positive protein identification rates for each said pair using randomizations of the peptide expression profiles; and for an optimal selection of the correlation coefficient threshold and peptide coverage threshold as determined by the false positive protein identification rate and number of proteins identified, generate a new peptide-to-protein assignment where all peptides assigned to a protein are pairwise correlated at or above the correlation coefficient threshold and the number of said peptides is at least the peptide coverage threshold.

In another aspect, there is a method of identifying biomolecules. The method may be performed by an automatic data processor and comprises: accessing data representing a plurality of expression patterns of biomolecule fragments expressed from one or more samples; using the accessed data, identifying at least one precursor biomolecule associated with said plurality of peptide expression patterns; determining a coefficient useable for measuring a correlation between a plurality of expression patterns of biomolecule fragments identified as associated with said precursor biomolecule; and based at least partly on the coefficient, identify at least one of a relatively high-confidence and a relatively low-confidence association of peptides with precursor proteins.

In another aspect, there is an apparatus useful for identifying proteins. The apparatus may comprise a data processor adapted to: access data representing a plurality of expression patterns of peptides expressed from one or more samples; using the accessed data, identify at least one protein associated with said plurality of peptide expression patterns; determine a coefficient useable for measuring a correlation between a plurality of expression patterns of peptides identified as associated with said protein; and based at least partly on the coefficient, identify at least one of a relatively high-confidence and a relatively low-confidence association of peptides with precursor proteins.

The plurality of peptide expression patterns may represent the expression of all peptides detected in a sample. The correlation coefficient may be determined only between expression patterns associated with peptides that are associated with a single protein. The processor may be adapted to access the data representing the expression patterns as signals provided by a liquid-chromatography/mass spectroscopy (LC-MS) analysis device. The processor may be adapted to access the data representing the expression patterns as signals recorded in persistent storage media. The persistent media may be associated with the data processor. The processor may be adapted to access the persistent media via a public communications network. The processor may be adapted to access the data representing the expression patterns as signals stored in volatile memory.

In another embodiment, there is an apparatus useful for identifying biomolecules. The apparatus may comprise a data processor adapted to: access data representing a plurality of expression patterns of biomolecule fragments expressed from one or more samples; using the accessed data, identify at least one precursor biomolecule associated with said plurality of biomolecule fragment expression patterns; determine a coefficient useable for measuring a correlation between a plurality of expression patterns of biomolecule fragments identified as associated with said precursor biomolecule; and based at least partly on the coefficient, identify at least one of a relatively high-confidence and a relatively low-confidence association of peptides with precursor proteins.

### BRIEF DESCRIPTION OF THE FIGURES

The foregoing and other aspects of the invention will become more apparent from the following description of specific embodiments thereof and the accompanying drawings which illustrate, by way of example only, the principles of the invention. In the drawings, where like elements feature like reference numerals (and wherein individual elements bear unique alphabetical suffixes):

FIG. 1 is a block diagram showing a process of bottom-up proteomics.

FIG. 2 is a block diagram showing a process flow of an embodiment of the invention.

FIG. 3 is a block diagram showing another process in the embodiment of FIG. 2.

FIG. 4 is a block diagram showing steps in a process in the embodiment of FIG. 2

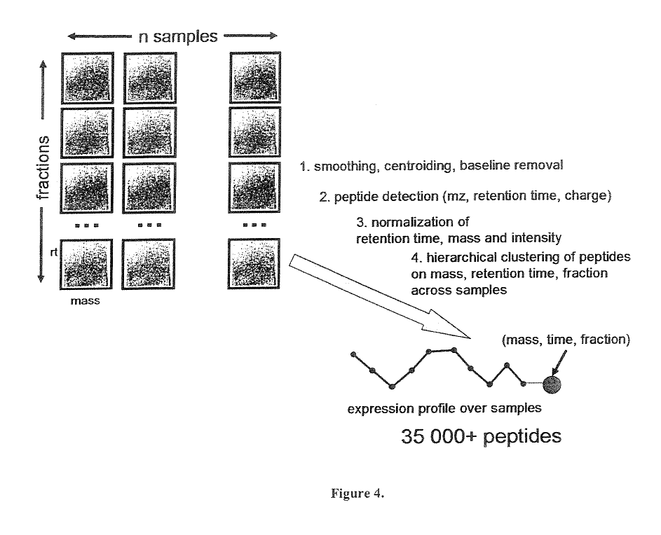

FIG. 5 is a block diagram showing a relationship between peptides and proteins in an embodiment.

FIG. 6 is a graph showing a correlation in an embodiment.

FIG. 7 is a matrix visualization graph of a correlation in an embodiment.

FIG. 8 is a visualization graph of a correlation in an embodiment.

FIG. 9 is a block diagram showing an alternate process in the embodiment of FIG. 2.

FIG. 10 is another visualization graph of another exemplary correlation in an embodiment.

FIG. 11 is yet other visualization graph of another exemplary correlation in an embodiment.

FIG. 12 is a chart of an exemplary correlation in an embodiment.

### DETAILED DESCRIPTION OF EMBODIMENTS

The description which follows, and the embodiments described therein, are provided by way of illustration of an example, or examples, of particular embodiments of the principles of the present invention. These examples are provided for the purposes of explanation, and not limitation, of those principles and of the invention. In the description, which follows, like parts are marked throughout the specification and the drawings with the same respective reference numerals.

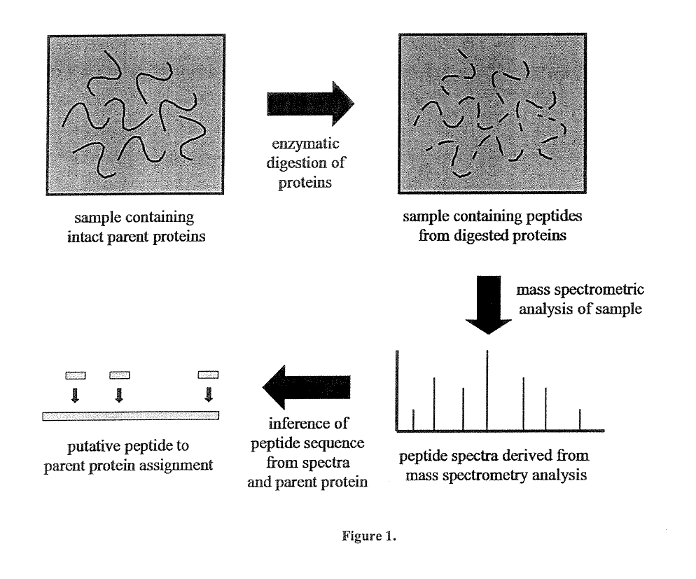

Bottom-up proteomics covers an approach to proteomics where biomolecules, such as proteins within a sample are digested using an enzyme such as trypsin resulting in a collection of peptides. The digested protein is generally referred to as the parent protein or precursor of the derived tryptic peptides. Protein identification in the context of bottom-up proteomics covers the assignment of peptides to parent proteins using proteomic technologies such as tandem mass spectrometry. The accuracy of protein identification is typically measured by the proportion of true positive to false positive parent protein identifications. See for example, FIG. 1 which shows a typical bottom-up proteomics analysis resulting in putative peptide-to-protein assignments.

Advantageously, in embodiments of the invention described below, protein identification in the context of bottom-up proteomics includes a procedure where a peptide-to-protein assignment is filtered by an independent procedure that differentiates the peptides likely to be true positive assignments from those likely to be false positive assignments. Furthermore, this procedure can tend to rigorously quantify the resulting false positive protein identification rate. The procedure, as used in protein identification, is referred to as PRotein IDentification and Expression (PRIDE).

Embodiments of the invention provides systems, methods, apparatus, and programming useful for improving the accuracy of peptide to biomolecule, or protein, assignments by utilizing expression profiles for each peptide and defining a procedure for determining the false positive rate of biomolecule identification.

More specifically, in an embodiment of the invention, there is taken as input a plurality of putative peptide-to-protein assignments and for each peptide an expression profile across a plurality of samples. The embodiment then measures the correlation of the expression profiles for each pair of peptides. A correlation threshold and coverage threshold are determined (as described in more detail below) and the largest set of peptides that have pairwise correlation coefficients, or scores, above a correlation threshold is selected as the correct peptide-to-protein assignments. If the size of this set of peptides is less than the coverage threshold then the protein is determine to be a false positive protein identification. The false positive protein identification rate is determined for multiple correlation and coverage threshold values, which enables the optimization of these two parameters so that the false positive protein identification rate can tend to be minimized, while tending to maximize the number of acceptable protein identifications.

Examples of technologies that generate peptide to biomolecule assignments include tandem mass spectrometry coupled with protein database search engines such as Mascot (Matrix Science, London, UK). Tandem mass spectrometry can also be coupled with de novo sequencing tools such as PEAKS (Bioinformatics Solutions, Waterloo, Canada) followed by protein homology searches. Fingerprinting tools such as Aldente (Expasy, Swiss Institute of Bioinformatics, Geneva, Switzerland) can be used also.

The peptide expression profiles used in the embodiment can originate from mass spectrometric analyses of biological or clinical samples including technologies such as MALDI, ESI and SELDI. Peptide expression levels across samples may also be measured using immunoassays or any other technology that quantifies peptide levels. ICAT and other labeling technologies can also generate peptide expression profiles (see for example Gygi, S P et al., supra).

Correlations between the pluralities of expression profiles of peptides may be determined using any suitable algorithm or method. Examples include the Pearson correlation, Spearman ρ correlation, Kendall's τ correlation, correlation ratio and mutual information, Gamma association, Stuaru's tau-c, and Somer's D correlations, as well as other widely-accepted standard definition employing least-squares curve fitting. See for example, Cohen, J. et al., supra.

The selection of the largest set of pairwise correlating peptides may be performed using various established algorithms including graph theoretic algorithms (largest clique) and hierarchical clustering.

The false positive rate of protein identification may be determined using methods such as permutation tests on the underlying expression data and other similar randomization techniques.

It is possible that peptides are related biochemically, but in general, are not biochemical related. For the embodiment, the only assumed relationship is that they originate from the same parent protein or biomolecule.

The embodiment does not require that any of the putative peptide-to-protein (or biomolecule) assignments be correct. In some instances, the procedure may find that none of the assigned peptides correlate.

This is based on the observation that peptides originating from the same protein or biomolecule precursor will tend to share the same expression profile across samples in a bottom-up proteomics study. This follows from the fact that the protein expression profile is determined in vivo before the proteins in the samples are digested (say, by trypsin) to obtain peptides.

A distinct but related concept is that peptides exhibiting correlated expression profiles are biochemically or biologically related will also exhibit correlation in vivo; see for example J. Lamerz et al., supra. This latter working assumption is the converse of the working theory upon which PRIDE and the embodiments are based. More specifically, a PRIDE system utilizes a peptide-to-protein assignment which associates peptides together because they are assigned to the same protein by a protein identification procedure. As applied in the embodiments, the PRIDE system confirms that these peptides have correlated expression profiles, or not.

Further details on particular embodiments of PRIDE is now provided. In analyses, the samples may include, for example, multiple samples taken from a single source, such as a human or animal patient or test subject, or samples taken from multiple human or other subjects, such as multiple patients in a clinical program or study. For example, multiple samples may be collected from healthy and diseased individuals.

As described herein, biomolecules include proteins, polypeptides, peptides, and carbohydrates. Biomolecule fragments include proteins, polypeptides, peptides, amino acids, carbohydrates, and any other portions into which biomolecules may be separated. The terms “peptide” and “parent protein” are well understood by a person of skill in the relevant arts and require no further elaboration.

A polypeptide include a chain of two or more amino acids, regardless of any post-translational modification (e.g., glycosylation or phosphorylation). Polypeptides include proteins and peptides. Source polypeptides may be cleaved by the action of a protease into one or more digestion fragments, or otherwise fragmented by any means compatible with the purposes disclosed herein.

A digestion fragment include a portion of a polypeptide produced, actually or theoretically, by for example the action of a protease or other agent that reproducibly cleaves or otherwise fragments the polypeptide.

A source polypeptide include a polypeptide from which a specified digestion fragment is actually or theoretically produced by, for example, the action of a protease or other chemical cleavage agent that reproducibly cleaves or otherwise fragments the source polypeptide. A source polypeptide typically contains at least two potential digestion fragments.

A fraction include a portion of an analyte or sample separation. A fraction may correspond to a volume of liquid obtained during a defined time interval, for example, as in LC (liquid chromatography). A fraction may also correspond to a spatial location in a separation such as a band in a separation of a biomolecule facilitated by gel electrophoresis, e.g., SDS-PAGE. Furthermore, a fraction may correspond to an elution from a chromatography medium, e.g., strong cation exchange.

In an embodiment, the pairwise correlation between ordered lists of values, X and Y, may be viewed as a measurement of the dependence between the two lists. That is, as values in X increase then the values in Y also increase. In a negative correlation, as values in X increase then values in Y decrease. If the dependence is linear then the pairwise correlation between X and Y is often measured using the Pearson correlation defined:

$${r}_{xy} = \frac{\sum ( {x}_{i} - x ) ( {y}_{i} - y )}{( n - 1 ) {s}_{x} {s}_{y}}$$

where xᵢ and yᵢ are the values of X and Y, x and y are the means and sₓ and s_{y} the standard deviations. The Pearson correlation tends towards 1 if there is a positive linear dependence and tends towards (−1) if there is a negative linear dependence. As the Pearson correlation tends to 0 there is no linear dependence between X and Y. As such, the Pearson correlation is an indication of the degree of linear dependence between X and Y. In the context of peptide expression profiles, the correlation between pairs of peptide expression profiles may be quantified using the Pearson correlation or other measures of dependence, as described below. In an embodiment, ordered lists of values such as X and Y can be log-transformed or normalized before quantifying the degree of dependence.

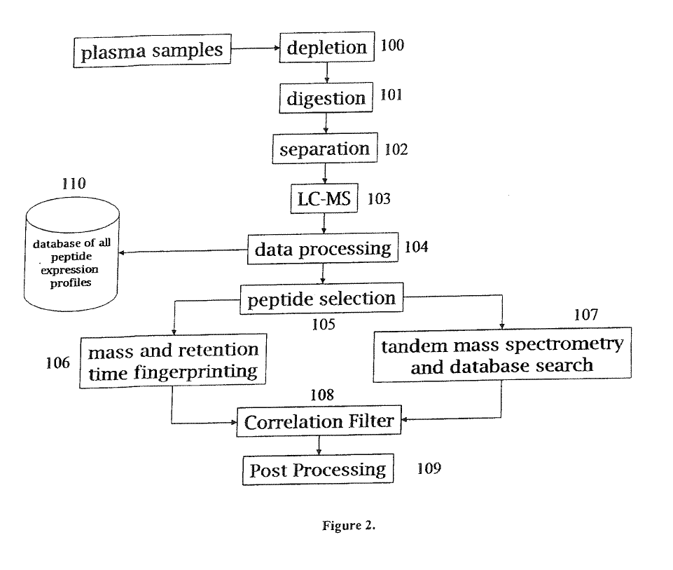

Referring now to FIG. 2, there is depicted a block diagram showing a process for identifying a biomolecule in accordance with an embodiment. The embodiment as described is implemented on a computer system, with elements including processor, data storage, and input/output devices and connections as known to a person of skill. While features of the embodiment are implemented in software on a computer readable medium, a person of skill, with reference to this description, can prepare the appropriate computer-readable code for a computer system on which the embodiment is implemented, and as such software code and pseudo-code is not provided herein. It will be appreciated that various hardware and/or software combinations may be used to implement different embodiments.

The embodiment of FIG. 2 shows a process flow where a sample being analyzed is plasma. However, it will be appreciated that any biological sample could be analyzed including, but not limited to, urine, cerebrospinal fluid, feces, saliva, biopsies, and others. Note that in a typical proteomic study 10's to 100's of samples are typically analyzed. At 100 of the process shown in FIG. 2, plasma samples are depleted of high abundance plasma proteins by an affinity column. The depleted sample then are moved on to digestion at 101. In the embodiment, digestion is generally accomplished enzymatically, e.g., by digestion with trypsin, elastase, or chymotrypsin. Other digestion may be used, such as digestion chemically, e.g., by cyanogen bromide. All samples that are to be compared are typically treated in the same manner.

After digestion there is an optional separation at 102. There are many separation technologies (see, for example, Laemmli, supra and Schagger et al., supra) including SDS-PAGE, SCX (Strong Cation Exchange), IEF (Isoelectric Focusing) among others. Such separation techniques are well known to a person of skill, and are therefore not repeated herein for brevity.

After separation, the fractions are submitted to a LC-MS analysis at 103. At 103, raw expression data is obtained for peptides. Exemplary methods for analyzing polypeptides and other biomolecules using mass spectrometry techniques are well known in the art (see for example, Godovac-Zimmermann et al., supra, Gygi et al. II, supra, Reinders et al., supra and Aebersold et al., supra), and doubtless others will hereafter be developed. The exact type of mass spectrometer used is not critical to the embodiments disclosed herein, and a person of skill will understand, with the descriptions herein, how to operate a mass spectrometer in accordance with the described embodiments.

Although the description of the embodiments herein are focused on polypeptides and other biomolecules, the embodiments are generally applicable to any biological polymers, e.g., oligosaccharides and polysaccharides, lipids, nucleic acids, and metabolites, capable of being detected via mass spectrometry.

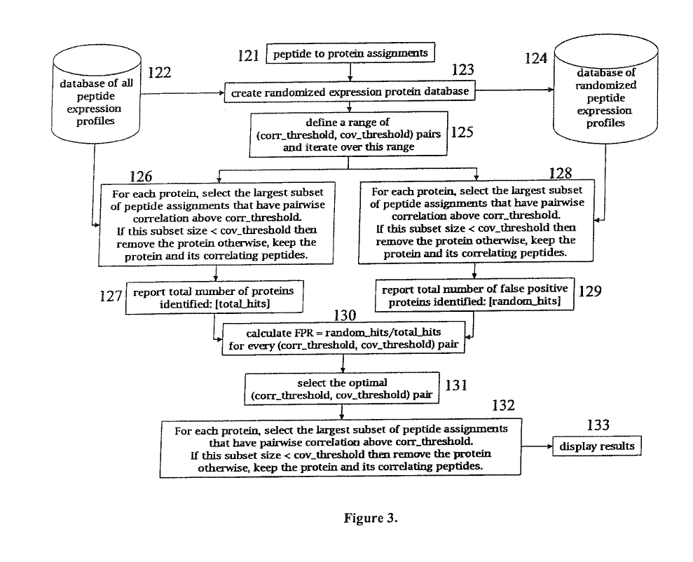

After the raw expression information is obtained in 103, at 104 the raw LC-MS data is processed in a series of refinements. Such processing of LC-MS raw data is shown in FIG. 3, which presents the data analysis process of the embodiment in more detail. FIG. 3 depicts a typical plasma proteomic study with n samples fractionated by SCX into multiple fractions. Each block in the figure represents the raw data obtained from an individual LC-MS injection. The raw data is smoothed, centroided and baseline removed. Most mass spectrometer software packages perform these basic functions such as MassLynx (Waters Corporation). Peptide detection is then performed, which determines the mass to charge (m/z) ratio, retention time and charge of each peptide's monoisotopic peak. In a typical analysis or study, there are approximately 5000 peptides per LC-MS injection detected. Software is used to perform peptide detection using the isotopic patterns of peptides, and examples of which are described in co-owned U.S. patent application Ser. No. 10/293,076 and filed 13 Nov. 2002, entitled “Mass Intensity Profiling System and Uses Thereof”. A commercial example of such software is Decon 2LS from Pacific North West national Labs.

Once peptides have been detected, three dimensions of LC-MS data, namely, mass, retention time and intensity, are normalized across the study. For the embodiment, this is accomplished by selecting a standard sample and normalizing to that sample. The next step of data processing is clustering. The goal of clustering is to track the same peptide, within a fraction, across all samples of the study. This is achieved by performing hierarchical clustering on mass and retention time for each fraction.

Referring back to FIG. 2, for the embodiment, the results of the analysis are stored in a database of peptide expression profiles (110) where each record has the form:
[Peptide_ID, fraction, m/z, retention time, charge, expression profile across n samples].
   This exemplary form of peptide expression patterns can then be used by the analysis techniques of the embodiment to identify a biomolecule, and to validate an identification of a biomolecule. It will be appreciated that other data storing methods, utilizing any data storage solution known in the art or developed hereafter, can be utilized for different embodiments.

Consequently, for the embodiment every peptide is assigned a unique identifier, the fraction it was detected in, the median m/z ratio and median retention time at which it was detected across the n samples of the study, the charge state and a vector representing the expression profile of the peptide across the study. In a typical plasma proteomic study with 8 SCX fractions, over 35000 highly reproducible peptides are typically found.

Returning to FIG. 2, after the data processing at 104 is completed and stored in 110, peptides of interest are selected for protein identification in process step 105. There are many criteria that may be used for selecting peptides of interest. For example, in a proteomic study comparing healthy and diseased plasma samples, peptides of interest are those that show a statistically significant difference between the healthy and diseased samples. Methods for selecting peptides include parametric and non-parametric tests, degree of differential abundance, AUC (area under the curve, of a receiver operating characteristic), intensity variability, and others. It will be appreciated that different peptide selection criteria may be used, depending on the study or biomolecule identification being conducted.

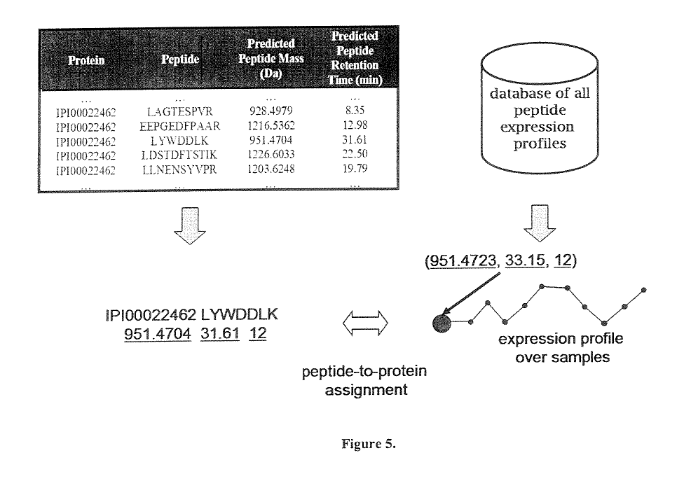

After peptides have been selected for biomolecule or protein identification, they are submitted to mass and retention time fingerprinting at 106, such as described in co-owned application No. 60/691,414, described and incorporated by reference above, and/or tandem mass spectrometry using LC-MS/MS followed by database searches using Mascot or some another search engine known in the art or hereafter developed at 107. Irrespective of the methodology used for biomolecule or protein identification, in the context of bottom-up proteomics as utilized in the embodiment, the resulting biomolecule or protein identification is an assignment of peptides in the peptide expression profile database to peptide sequences within a parent biomolecule or protein. A graphical representation of an exemplary association is depicted in FIG. 5. Therein, note that there can be multiple peptides assigned to each protein or biomolecule, and each peptide can be assigned to multiple proteins or biomolecules. The latter assignment is understood to be a consequence of the non-specificity of peptide assignments to proteins or biomolecules.

After protein identification is completed at 106 and/or 107, the results of such protein identification efforts are merged and sent to a correlation filter 108, as shown in FIG. 2. The details of the correlation filter of the embodiment is shown and described in more detail with reference to FIG. 3. In the embodiment, the correlation filter is implemented in computer software to provide a confidence assessment of the peptide to biomolecule assignment. It will be appreciated that the filter can be implemented in other hardware and/or software combinations in other embodiments.

Referring to FIG. 3, peptide to protein (or other biomolecule) assignment at 121 is provided with data 122. For the embodiment, data 22 may be based on, or be an exact copy, of data 110. At 123, the correlation filter creates a randomized peptide expression data set 124 from a peptide expression profile database 122. For the embodiment, this is achieved by randomizing the association of peptides to expression profile vectors, and/or by randomizing the order of the peptide expression profile vector for each peptide in the database. As described below, this randomized data set 124 is used in the embodiment to help identify by-chance associations of biomolecules to peptides detected in a sample under analysis. A peptide expression profile database 122 may be populated by data found by a user of the PRIDE system, or the data may be obtained from another source for use in the system. At 125, the correlation filter defines two parameters, namely, the correlation threshold and the coverage threshold: corr_threshold and cov_threshhold. At 125, a range of values is defined for these two parameters from which an optimal pair of values will be determined. As described below, the values of these parameters are used in an embodiment as a correlation coefficient in determining correlations. This feature is further illustrated in Example 2, below.

To select the corr_threshold parameter in a study independent manner, it is represented as a percentile value rather than an absolute correlation value. The reason for this choice in the embodiment is that peptide expression correlation coefficients are dependent upon the number of samples analyzed and the variability of the underlying proteomic platform. To obtain a percentile value, the distribution of all pairwise correlation coefficients between pairs of peptides in the database is determined using, for example, the Pearson correlation (or some other correlation method known or hereafter known in the art). This distribution can then be used to determine the percentile value of any raw correlation coefficient. Since a raw correlation score depends on, among other factors, the number of samples in the study, the inherent variability of the proteomic platform and the samples analyzed, converting to a percentile standardizes the approach used in the embodiment to determine confidence. This is tends to be advantageous as it enables comparisons among studies, which comparisons have heretofore not been seen in such studies.

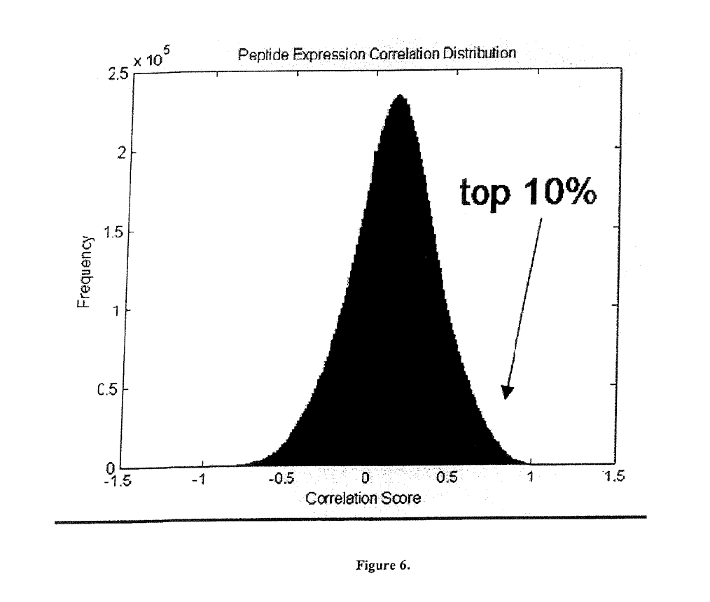

Referring to FIG. 6, there is shown an example of a correlation distribution of pairwise Pearson correlation scores. The corr_threshold value is selectable from a range of values. In this example shown, the corr_threshold may be set to the correlation score representing the 90th percentile of the distribution. The value of the 90th percentile can be changed from study to study, and therefore, the use of a percentile normalizes the choice of corr_threshold across multiple studies.

For example, the Pearson correlation for two sets of measurements X and Y is defined:

$${r}_{xy} = \frac{\sum ( {x}_{i} - x ) ( {y}_{i} - y )}{( n - 1 ) {s}_{x} {s}_{y}}$$

where xᵢ and yᵢ are the values of X and Y, x and y are the means and sₓ and s_{y} the standard deviations. The Pearson correlation tends towards 1 if there is an increasing linear relationship and tends towards (−1) if there is a decreasing linear relationship. As the Pearson correlation tends to 0 there is no linear relationship between X and Y. As such, the Pearson correlation is an indication of the degree of linear dependence between X and Y.

The Pearson correlation is a parametric statistic. If the measurements X and Y are not normally distributed, then non-parametric correlation metrics such Spearman's ρ and Kendall's τ can be used. Even more general correlation measures that may be applied are the correlation ratio and mutual information. The mutual information of measurements X and Y is defined:

$$M ( X , Y ) = {\sum }_{x ∈ X} {\sum }_{y ∈ Y} p ( x , y ) log \frac{p ( x , y )}{p ( x ) p ( y )}$$

where p(x,y) is the joint probability distribution of X and Y, and p(x) and p(y) are the marginal probabilities of X and Y. Mutual information measures how much is known about Y if X is known, or vice-versa.

Although standard measures of correlation or dependence between measurements X and Y are utilized in the embodiments described, any measurement of correlation or dependence can be used in other embodiments that produces a coefficient that quantifies the degree of correlation or dependence.

Referring back to FIG. 3, at 126 each biomolecule and all peptides assigned to that protein are analyzed. For the embodiment, the peptides are clustered using average linkage hierarchical clustering where the inter-peptide distance metric used for the clustering is (1−P_{xy})/2 where P_{xy} is the percentile Pearson correlation coefficient for peptides x and y. This transforms the Pearson correlation into a distance metric that ranges from 0 to 1. The resulting cluster tree is traversed and the subtree with the largest number of peptides with pairwise correlation scores below corr_threshold is determined. If the number of peptides in this subtree is less than cov_threshold (i.e. less than the required coverage) then the biomolecule is removed from the list of identified proteins. Otherwise, the biomolecule and the peptides in the subtree are kept. All other assigned peptides to this biomolecule are removed. Hierarchical clustering is one of many algorithms that could be used to find a subset of correlated peptides in different embodiments.

Another approach that may be used include graph theoretic approaches such as finding the maximum clique in a graph (see Garey et al., supra), where each node in the graph is a peptide, and there is an edge between pairs of peptides if their percentile Pearson coefficient is below corr_threshold. Other methods of finding a maximal set of correlating peptides may be used in other embodiments. As described above and below, a wide variety of existing statistical methods may be employed in assessing the significance of correlations. Some such statistical methods may be based, for example, on varying assumptions related to interpretation of the fragment expression patterns, the propriety of the various assumptions and therefore of the use of the various statistical methods depending upon the nature and purpose of the fragment-precursor studies, and the techniques employed therein. Examples of suitable algorithms include the Pearson correlation, Spearman rank correlation, Kendall's rank correlation, Gamma association, Stuaru's tau-c, and Somer's D correlations, as well as other widely-accepted standard definition employing least-squares curve fitting.

Thus, at 126 for each protein identified in the initial peptide-to-protein assignment, the largest subset of peptide assignment that have pairwise correlation above the correlation threshold is determined. If the subset size, i.e., the number of peptides assignments having pairwise correlation above the correlation threshold, is less than the coverage threshold value, then the biomolecule is removed from the list of identified proteins. Otherwise, the biomolecule and its corresponding peptides are kept. In the embodiment, the kept biomolecule and its corresponding peptides can be considered a relatively high-confidence association, while the removed biomolecule and its corresponding peptides can be considered a relatively low-confidence association. Of course, it will be appreciated that such associations are variable with the correlation coefficient that is selected for the particular analysis.

It will also be appreciated that correlation coefficients can be preset, or determined during an analysis as described above. Until a coefficient is selected as optimal at 131, the correlation coefficients used in the determinations may be considered test coefficients.

Referring back to FIG. 2, after the proteins or biomolecules have been processed at 126, the total number of proteins remaining is determined (total_hits) at 127. To estimate the false positive rate, the process of 126 and 127 is repeated (by way of 128 and 129), but now a database of randomized peptide expression profiles 124 is used instead to determine any by-chance associations of biomolecule-to-peptide(s) assignments That is, the same range of parameter values for corr_threshold and cov_threshold are used, but this time with a view to determining an expected random correlation and false identifications based on by-chance peptide-to-biomolecule associations. Thus, the number of proteins, or biomolecules, that remain after process step 129 (random_hits), at 131, is the number of proteins expected to pass the correlation filter by chance alone. This is the case because peptides will be correlated only by chance since their expression profiles are random. Consequently, the false positive rate (FPR) is equal to random_hits divided by total_hits. As shown at 130, each pair of parameter values in the range is assessed is assigned a FPR based on the particular corr_threshold and cov_threshold pair. This randomization procedure can be iterated numerous times for each pair of parameter values in the range and then an average number of random_hits over the iterations may be used as an even more robust estimate of the number of false positives.

At 131, the false positive rate and the total number of proteins identified (at 127 for non-randomized determination by 126) are considered. Depending on the requirements of a particular application, a low false positive rate might be required due to the cost or risk of permitting a false positive protein identification. Other applications may be more tolerant to errors and will thus accept a higher false positive rate in exchange for more proteins identified. Based on the contextual goals of a particular analysis, for an embodiment at 131 optimal values for corr_threshold and cov_threshold can be selected. In an embodiment, considerations might be to select the corr_threshold and/or cov_threshold values that are higher (to decrease the false positive rate) or lower (to increase the total number of proteins identified).

Referring back to FIG. 3, at 132, the peptide to biomolecule, or protein, assignment is produced based on a selected correlation coefficient, and at 133, the results of the correlation filter are displayed. In this way, a biomolecule identification may be validated by the embodiment, in that the identification of any biomolecule is considered to be validly correlated one or more peptide-to-biomolecule assignment within an error tolerance (such as a false positive identification rate) of the analysis being conducted.

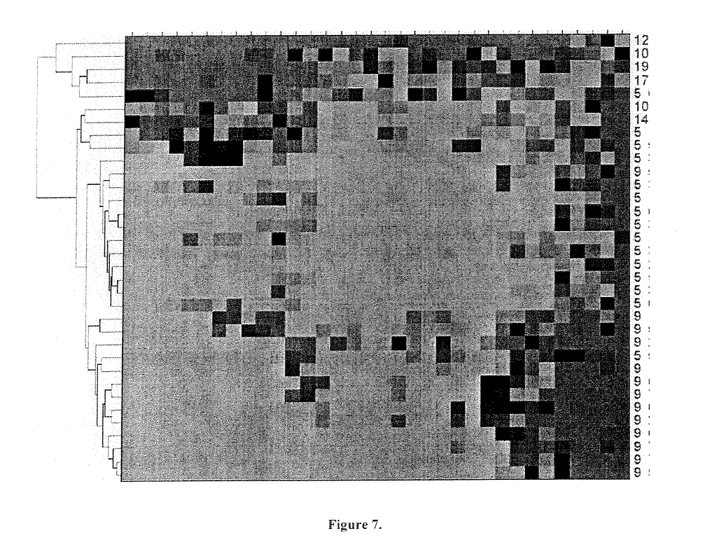

Displaying at 133 is typically done via a display unit at a computer terminal, but it will be appreciated that other outputs are possible. Visualization of the correlations among a set of peptides assigned to a protein or biomolecule are generally helpful for manual inspection. For example, in FIG. 7, the peptides assigned to an exemplary protein by LC-MS/MS index the rows and columns of a light-dark matrix. The matrix square indexed by two peptides (i.e. a peptide from a row and from a column) has a shade proportional to the degree of correlation. Correlation coefficients decrease from light through to dark. On the left of the matrix is the results of hierarchical clustering applied to the correlation matrix and on the right of the matrix is a column of numbers, one for each peptide, indicating the SDS-PAGE band from which the protein was identified. In this visualization, it becomes apparent to a person of skill which peptides are well-correlated both pairwise and as a group. As shown, peptides that are not well-correlated (for example peptides with dark shading) are clearly not correlated and are thus likely false assignments to the parent protein. Finally, there are groupings of peptides from SDS-PAGE band 5 and band 9 indicating that the parent protein has been either proteolysed, modified or is detected in two splice variants.

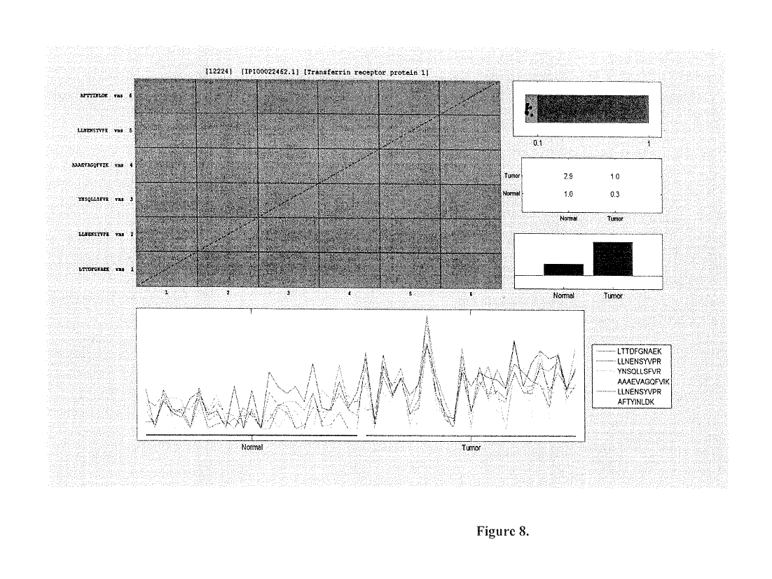

Another example appears in FIG. 8. Six peptides have been assigned to a parent protein and appear in the lower right legend. The expression profiles for these six peptides across 25 normal and 25 tumor samples, as shown, were measured by reverse phase liquid chromatography linked to an electrospray ion source Q-TOF mass spectrometer. These six expression profiles appear in the lower pane. Visually, the correlation pattern of these six peptides can be seen to be correlated. In the upper left pane, the pairwise correlation between pairs of peptides is visualized by a light-dark matrix such as in FIG. 7 above. Non-correlating peptides have been filtered out leaving a predominantly light matrix. In the upper right pane is the percentile score for each pair of peptide correlation coefficients as measured against the distribution of all pairwise peptide correlation coefficients in the study. For the embodiment, all pairwise peptide correlation coefficients appear in the top 10% (i.e. 90th percentile) of all peptide correlation scores. The average differential abundance of the tumor samples relative to the normal samples appears in the middle two panes on the right of FIG. 7.

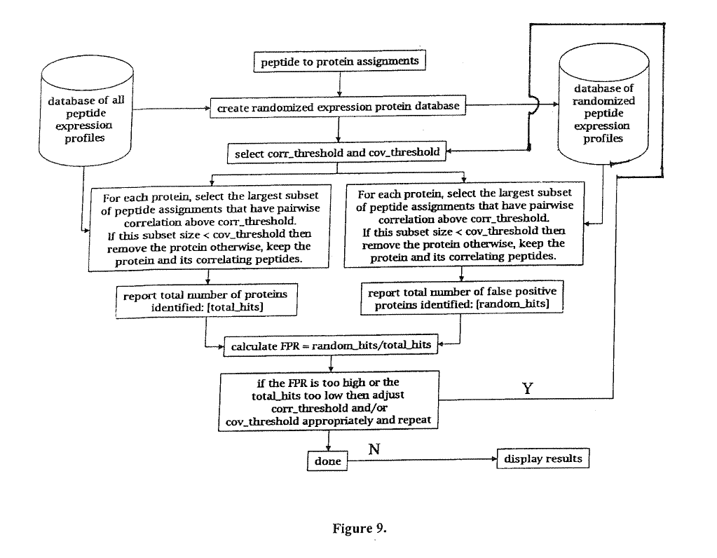

In another embodiment of the correlation filter, the correlation threshold and coverage threshold pairs that is acceptable can be determined iteratively. For example, the correlation threshold can be initially set to 90th percentile of the distribution, and the resulting FPR calculated therewith. The FPR and result set are examined to see if they are acceptable, and the correlation threshold and coverage threshold can be adjusted accordingly. For instance, in an embodiment, if one desires the FPR to be decreased, then corr_threshold and cov_threshold values can be adjusted upward; and if one desires that the total number of proteins identified be increased, then corr_threshold and cov_threshold can be adjusted downward. An example of such an iterative coefficients selection process is shown in FIG. 9.

In other embodiments, simplified filtering may also be applied so that if a biomolecule does not have enough matches for its size, then it may be eliminated from further consideration. Other filters may further include restricting polypeptides accepted by their size, raw number of hits, and/or other scoring criteria.

Returning to FIG. 2, the final step in the described embodiment is post processing at 109. This may include clustering of homologous identified proteins or biomolecules, ensuring that peptides are assigned to one protein or biomolecule only, annotation of proteins or biomolecules with GO terms, detection of functional domains, and other processing that might be desirable.

The results displayed at 130 relating to correlation coefficients can be used for a variety of purposes, depending upon the goals of the analysis. For example:
low-confidence correlations can be used to exclude peptides from further analysis of biomolecules of interest;
resolution or clarification of previously ambiguous fragment-precursor associations (e.g., in cases where single fragments are identified as children of multiple precursors); the precursor identified as correct can be that for which the fragments best correlate to each other;
delineation of splice variants, polymorphisms, and/or homologous proteins or other precursors. Multiple groups of fragments identified as children of a single precursor despite having different expression patterns may be correlated within the various groups. This can suggest the existence of splice variant, polymorphic, or homologous precursors. If two or more precursor biomolecules share similar fragments, then expression patterns associated with the fragments can be de-convoluted into their component profiles, and thus support multiple-precursor hypotheses;
assignment of confidence scores associated with parent-child identifications. Common biomolecule identification confidence scores include MOWSE (mass fingerprinting) and/or Mascot®/Sequest® (tandem mass spectrometry) as described above. Expression fragment correlation can provide entirely orthogonal methods of measuring confidence in precursor identification.
enablement of low-specificity precursor identification methods. Techniques such as tandem mass spectrometry can provide high-confidence precursor identifications with relatively few fragment spectra (i.e., low fragment coverage), whereas techniques such as mass fingerprinting can require relatively larger amounts of spectra data to make identifications of similar levels of confidence. Incorporation of fragment expression pattern correlation into methods such as mass fingerprinting can enable improved confidence with reduced amounts of fragment data. This is a direct consequence, for example, of the observed fact that at 1% significance, the probability of three fragments being erroneously identified as children of a precursor by mass, and being correlated, is less than 1/10,000.
correlation of fragment expression patterns with clinical profiles. For example, peptide expression patterns can also be correlated to profiles generated from sources of information other than mass spectrometry. For example, peptide expression profiles can be correlated to clinical data such as gender, age, disease stage, drug treatment, etc.
can implement a subsequent correlation, as for example by correlating precursor or parent biomolecules identifications to clinical data, conditions, or clinical outcomes.

### EXAMPLE 1

### Brucella Virulence Analysis

As example, the analysis of brucella virulence is examined below. Brucella virulence is linked to components of the cell envelope and tightly connected to the function of the BvrR/BvrS sensory-regulatory system. In this example, a label-free mass spectrometry-based analysis of spontaneously released outer membrane fragments from four strains of Brucella abortus: wild type virulent, avirulent bvrR− and bvrS− mutants as well as reconstituted virulent bvrR+ was performed to quantify the impact of BvrR/BvrS on cell envelope proteins. In total 167 differentially expressed proteins were identified of which 25 were assigned to the outer membrane.

Six samples of each strain were analyzed using the embodiment depicted in FIG. 2, except that depletion and separation were not performed. Full details of the background to the example is available in Lamontagne, et al., Extensive cell envelope modulation is associated with virulence in Brucella abortus, supra.

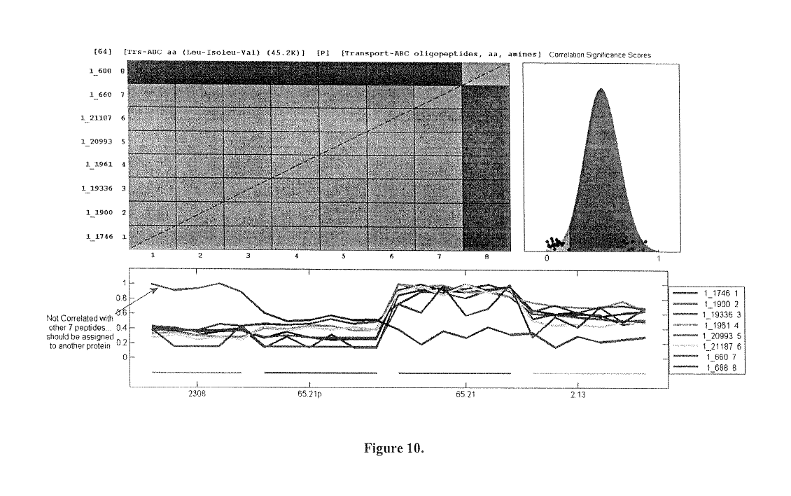

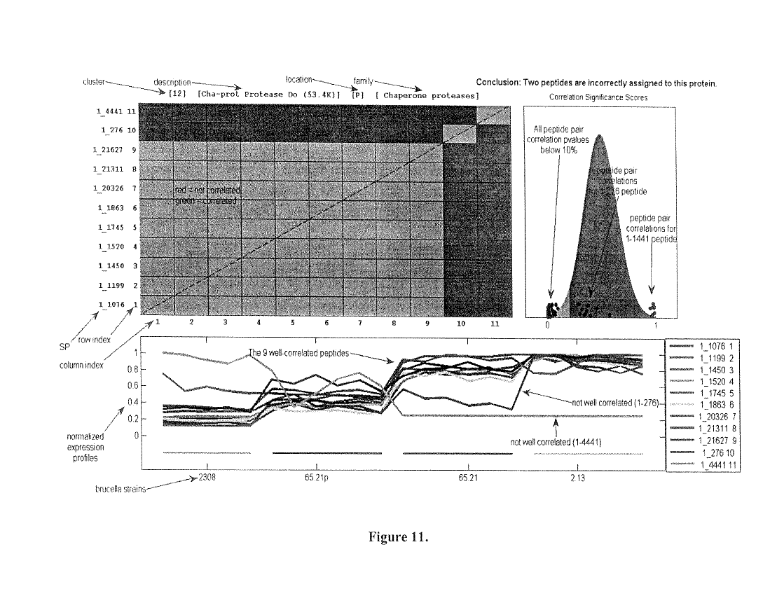

To increase confidence in the protein identification results and to decrease the possibility of wrongly assigned peptides, the correlation filter as described with reference to FIG. 3 was applied to all identified proteins and their expression profiles. The expression profiles for each peptide were obtained in accordance with 103 to 104 of the process presented in FIG. 2, and stored in a peptide expression profile database (110 in FIG. 2). To illustrate the results, two protein identifications are depicted in FIGS. 10 and 11 (the results in FIG. 11 is described in relation to Example 2, below). Note that there are many different peptide expression profiles as a results of the underlying biology and study design. However, the working theory is that peptides originating from the same protein will have correlated expression profiles since protein digestion into peptides occurs ex vivo. In both cases, nearly all assigned peptides have highly correlated expression profiles over the 24 samples in the study. However, in each case, at least one peptide has a completely different expression profile suggesting that this peptide has been wrongly assigned. As can be seen in FIG. 10, the peptides in this example are highly correlated except for peptide 1_{—}688, while the expression profile across the four Brucella strains (2308, 65.21p, 65.21 and 2.13) is clearly distinct from the other assigned peptides. Consequently, peptide 1_{—}688 can be deemed to be a false positive assignment. Note however that this does not diminish the confidence in the protein identification because there are still many correlated peptides assigned to this protein. However, the there is an increase in the confidence of the peptide-to-protein assignment(s) since false positive peptide assignments have been removed. In FIG. 11, two peptides, namely 1_{—}276 and 1_{—}4441, are visually and quantitatively different from the remaining peptides and the conclusion is that they are false positive peptide-to-protein assignments.

### EXAMPLE 2

### Prostate Plasma Analysis

In another example, 24 Healthy and 24 Prostate cancer plasma samples were analyzed using the process depicted in FIG. 2, except that protein identification was performed using mass and retention time fingerprinting only (i.e. tandem mass spectrometry was not performed). This resulted in a putative list of 427 peptides assigned to 2649 proteins where the mass and retention time matching tolerances were 25 ppm and 2.5 minutes (10% of total elution time). With an expected coverage of 2 peptides per protein, the expected number of true proteins identified would be approximated 213. With an expected coverage of 3 peptides per protein, the expected number of true proteins identified would be approximately 142. Clearly, there is a strong likelihood of a large number of false positive peptide-to-protein assignments. False peptide-to-protein assignments were then filtered out using the correlation filter as described in relation to FIG. 3. In the example shown in FIG. 11, the peptides are highly correlated across the four strains except for peptides 1_{—}4441 and 1_{—}276, which can be deemed false assignments.

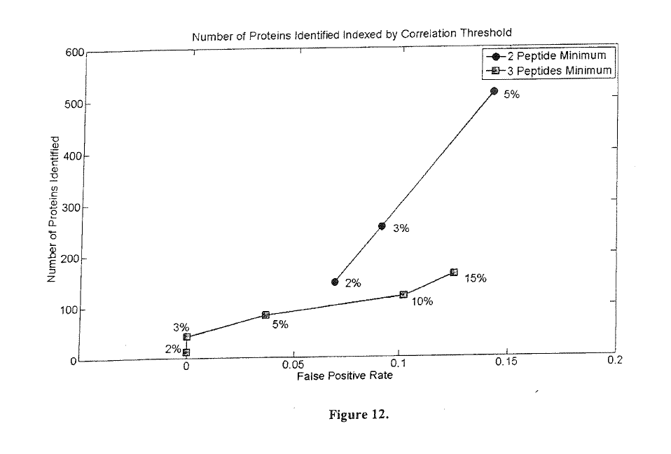

The process shown in FIG. 3 is applied using corr_threshold and cov_threshold pairs of (2%, 2), (3%, 2), (5%, 2), (2%, 3), (3%, 3), (5%, 3), and (15%, 3). The resulting number of false positive protein identifications and total protein identifications in this example appear in FIG. 11. Given that the expected number of correct protein identifications with coverage 2 and 3 are 213 and 142, respectively, the correlation and coverage threshold pairs of (2.5%, 2) and (10%, 3) both produce the expected number of protein identifications and with reasonable false positive protein identification rates (below 10%). Note that the pair (2.5%, 2) is an interpolation of the (2%, 2) and (3%, 2) values in FIG. 11. Given that (2.5%, 2) generates a lower false positive rate and more protein identifications than (10%, 3), according to the results of FIG. 12, it is the preferred choice of parameters for generating the final result as defined in 131 and 132, with reference to FIG. 3.

While the foregoing invention has been described in some detail for purposes of clarity and understanding, it will be appreciated by those skilled in the relevant arts, once they have been made familiar with this disclosure, that various changes in form and detail can be made without departing from the true scope of the invention in the appended claims. The invention is therefore not to be limited to the exact components or details of methodology or construction set forth above. Except to the extent necessary or inherent in the processes themselves, no particular order to steps or stages of methods or processes described in this disclosure, including the Figures, is intended or implied. In many cases the order of process steps may be varied without changing the purpose, effect, or import of the methods described.

What is claimed is:

1. A method useful in an identification of proteins, the method performed by a data processor and comprising:
accessing data representing a plurality of expression patterns of peptides expressed from one or more samples;
using the accessed data, identifying at least one protein associated with the plurality of peptide expression patterns;
selecting a correlation coefficient useable for determining a correlation between each at least one protein and a plurality of expression patterns of peptides identified as associated therewith; and
using at least the correlation coefficient, identifying at least one of a relatively high-confidence association and at least one of a relatively low-confidence association of precursor proteins with the peptides expressed from the one or more samples.

2. The method of claim 1, wherein:
the correlation coefficient includes a correlation threshold value and a coverage threshold value; and
the identifying the at least one relatively high-confidence and low confidence associations of precursor proteins include:
identifying a largest subset of the plurality of expression patterns associated with the each at least one protein, the subset having pairwise correlation above the correlation threshold value; and
identifying the each at least one protein as (i) a at least one relatively high-confidence association of precursor proteins if the subset size is greater or equal to the coverage threshold value, and (ii) a at least one relatively low-confidence association of precursor proteins if the subset size is small than the coverage threshold value.

3. The method of claim 2, further comprising:
accessing second data representing randomized expression patterns of peptides; and
using at least the correlation coefficient, identifying from the second data at least one of a relatively high-confidence by-chance association and at least one of a relatively low-confidence by-chance association of the at least one proteins with the peptide expressed from the one or more samples, by:
identifying in the second data a largest subset of the plurality of expression patterns by-chance associated with the each at least one protein, the subset having pairwise correlation above the correlation threshold value; and
identifying the each at least one protein as (i) a at least one relatively high-confidence by-chance association if the subset size is greater or equal to the coverage threshold value, and (ii) a at least one relatively low-confidence by-chance association if the subset size is small than the coverage threshold value.

4. The method of claim 3, further comprising determining a false positive rate as a ratio of a total of the at least one relatively high-confidence association of the precursor proteins over a total of the at least one relatively high-confidence by-chance association of the at least one proteins with the peptide expressed from the one or more samples.

5. The method of claim 4, further comprising evaluating whether the false positive rate is unacceptable, and if it is unacceptable, then selecting a new correlation threshold to replace the correlation threshold for use in repeating the said identifying steps until the false positive rate is acceptable.

6. The method of claim 5, wherein the expression patterns are obtained by liquid-chromatography/mass spectroscopy (LC-MS) analysis.

7. The method of claim 5, wherein the data relating to each expression pattern is obtained by digesting a corresponding peptide with a protease.

8. The method of claim 5, wherein the accessing data representing the pluralities of expression patterns of peptides comprises accessing data obtained using mass spectrometry.

9. The method of claim 5, wherein the accessing data representing the pluralities of expression patterns samples comprises accessing data obtained using virtual mass spectrometry.

10. The method of claim 5, wherein the data representing the plurality of expression patterns of peptides expressed from the one or more samples is accessed at least in part from real time analysis by a mass spectroscopy device associated with the processor.

11. A method of validating a biomolecule identification from a plurality of peptides, comprising:
using at least an assignment of the plurality of peptides to at least one precursor biomolecule from a set of peptide expression profiles, determining a correlation coefficient for correlating the assignment of the plurality of peptides to the at least one precursor biomolecule within a false positive identification rate; and
validating the biomolecule identification based on the assignment, if the biomolecule identification is correlated to one or more of the at least one precursor biomolecule within the false positive identification rate.

12. The method of claim 11, wherein the false positive identification rate is determined as a function of an expected random correlation between the plurality of peptides to the at least one biomolecule within the set of peptide expression profiles.

13. The method of claim 12, wherein:
the expected random correlation is a total number of expected false identifications based on the at least one biomolecule; and
the false positive identification rate is determined as a ratio of the total number of expected false identifications over a total number of identifiable biomolecules, the total number of identifiable biomolecules being based on the at least one biomolecule.

14. The method of claim 13, wherein:
the correlation coefficient comprise a correlation threshold and a coverage threshold;
the total number of identifiable biomolecules is determined by, for each of the at least one biomolecule, incrementing the total number of identifiable biomolecules if, in the set of peptide expression profiles, a largest subset of peptide assignment to the each at least one biomolecule has pairwise correlation above the correlation threshold and the subset has a size above the coverage threshold; and
the total number of expect false identifications is determined by, for each of the at least one biomolecule, incrementing the total number of expected false identifications if, in a randomized set of peptide expression profiles, another largest subset of peptide assignment to the each at least one biomolecule has pairwise correlation above the correlation threshold and the subset has a size above the coverage threshold, the randomized set of peptide expression profiles being generated from the set of peptide expression profiles.

15. The method of claim 14, wherein the correlation coefficient is selected on the basis of the false positive identification rate.

16. The method of claim 15, wherein the biomolecule is a protein.

17. The method of claim 16, wherein:
the correlation coefficient is selected from a plurality of test correlation coefficients, each of the test correlation coefficients being used to calculate a respective test false identification rate in the same manner that the correlation coefficient is used to determine the false positive identification rate, and
the test correlation coefficient having a test false identification rate that is closest within the false positive identification rate is selected as the correlation coefficient.

18. The method of claim 16, wherein the correlation coefficient is selected by initially selecting a test correlation coefficient to determine a test false identification rate in the same manner that the correlation coefficient is used to determine the false positive identification rate, and if the test false identification rate is not within the false positive identification rate, iteratively adjusting the test correlation coefficient until the test false identification rate is within the false positive identification rate, and then selecting the test correlation coefficient as the false positive identification rate.

19. A computer usable medium having computer readable code embodied therein for causing a computer to:
access data representing a plurality of expression patterns of peptides expressed from one or more samples;
using the accessed data, identify at least one protein associated with the plurality of peptide expression patterns;
select a correlation coefficient useable for determining a correlation between each at least one protein and a plurality of expression patterns of peptides identified as associated therewith, the correlation coefficient having a correlation threshold value and a coverage threshold value; and
using at least the correlation coefficient, identify at least one of a relatively high-confidence association and at least one of a relatively low-confidence association of precursor proteins with the peptides expressed from the one or more samples, by:
identifying a largest subset of the plurality of expression patterns associated with the each at least one protein, the subset having pairwise correlation above the correlation threshold value; and
identifying the each at least one protein as (i) a at least one relatively high-confidence association of precursor proteins if the subset size is greater or equal to the coverage threshold value, and (ii) a at least one relatively low-confidence association of precursor proteins if the subset size is small than the coverage threshold value.

20. The computer usable medium of claim 19, wherein the computer readable code embodied therein further causes the computer to:
access second data representing randomized expression patterns of peptides;
using at least the correlation coefficient, identify from the second data at least one of a relatively high-confidence by-chance association and at least one of a relatively low-confidence by-chance association of the at least one proteins with the peptide expressed from the one or more samples, by:
identifying in the second data a largest subset of the plurality of expression patterns by-chance associated with the each at least one protein, the subset having pairwise correlation above the correlation threshold value, and
identifying the each at least one protein as (i) a at least one relatively high-confidence by-chance association if the subset size is greater or equal to the coverage threshold value, and (ii) a at least one relatively low-confidence by-chance association if the subset size is small than the coverage threshold value;
determine a false positive rate as a ratio of a total of the at least one relatively high-confidence association of the precursor proteins over a total of the at least one relatively high-confidence by-chance association of the at least one proteins with the peptide expressed from the one or more samples; and
evaluate whether the false positive rate is unacceptable, and if it is unacceptable, then selecting a new correlation threshold to replace the correlation threshold for use in repeating the said identifying steps until the false positive rate is acceptable.
